(19) United States
(12) Patent Application Publication
(10) Pub. No.: US 2014/0100503 A1
(43) Pub. Date: Apr. 10, 2014

(54) LUMBAR TRACTION DEVICE

(71) Applicant: Chung-Fong LIAO, New Taipei City (TW)

(72) Inventor: Chung-Fong LIAO, New Taipei City (TW)

(21) Appl. No.: 13/648,714

(22) Filed: Oct. 10, 2012

(51) Int. Cl. A61F 5/04

(52) U.S. Cl. 602/36

(57) ABSTRACT

A lumbar traction device includes a tabular base, an indentation, a lumbar tracking base, tightening pieces, a weight member, a driving member and a controller. The lumbar tracking device allows the user to grip the controller to control the rotation of the rotation axis, and drive the threaded rod to rotate spirally forward and backward so as to synchronously drive the lumbar tracking base to move forward and backward for stretching or releasing. Hence, the user can independently operate the lumbar traction device for optimal applicability and safety. The weight member is further provided with a weight adjustment component to adjust the weight levels required by the user for lumbar traction. A warning part emit warning signals to inform the user that the preset weight value is reached, helping to avoid discomfort or secondary injuries resulting from overload.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to a body traction device, and more particularly to an innovative structural design of a lumbar traction device.

2. Description of Related Art Including Information Disclosed Under 37 CFR 1.97 and 37 CFR 1.98.

There are many reasons causing pain or discomfort in the lumbar or lower back part. In the case of young people, the main reasons include: long-term poor posture, carrying heavy weights, sports injury etc., may cause herniated disk and nerve compression, and consequently cause pain in the lumbar or lower back part. In serious cases, the whole lower part of the body (such as buttocks and legs) can be affected. In the case of middle-aged and old people, apart from long-term poor posture, reasons also include lumbar degenerative arthritis or hyperosteogeny which lead to nerve compression and pain. Therefore, both young and old people are prone to this kind of disease.

To solve the above-mentioned problems, general hospitals will usually adopt a rehabilitation method of physical traction in addition to hot compress or prescription to obtain a good curing effect. The conventionally known lumbar traction mechanism roughly includes a rehabilitation bed and a traction device provided on the rehabilitation bed. When the patient is lying on the rehabilitation bed, the medical personnel manually moves the traction device to stretch and release the lumbar part of the patient's body. However, a problem of the conventional structure is found in actual applications: as the affected part of each patient can only withstand different strengths, but the conventional lumbar traction mechanism can only be driven manually by the medical personnel, the patient may suffer from overload but fail to tell the medical personnel timely, leading to bad rehabilitation effect or even secondary injury.

Moreover, as there is no appropriate resistance in the driving of the conventional lumbar traction mechanism, speedy stretching or releasing may cause discomfort or injury in the affected part. Hence, some improvements are necessary.

Thus, to overcome the aforementioned problems of the prior art, it would be an advancement if the art to provide an improved structure that can significantly improve the efficacy.

Therefore, the inventor has provided the present invention of practicability after deliberate design and evaluation based on years of experience in the production, development and design of related products.

### BRIEF SUMMARY OF THE INVENTION

Based on the innovative structural design of the “lumbar traction device” disclosed in the present invention wherein the lumbar traction device comprises as tabular base, an indentation, a lumbar tracking base, tightening pieces, a weight member, a driving member and a controller, the present invention surpasses the conventionally known structure described in prior art by enabling the user to grip the controller to control the rotation of the rotation axis, and drive the threaded rod to rotate spirally forward and backward so as to synchronously drive the lumbar tracking base to move forward and backward for stretching or releasing. Hence, the user can independently operate the lumbar traction device for optimal applicability and safety.

The present invention can have the following new efficacies:

Based on the technical characteristic wherein the weight member is further provided with a weight adjustment component to provide a function to adjust the weight levels required by the user for lumbar traction, and through configuration of a warning part to emit warning signals to inform the user that the preset weight value is reached, the present invention can help to avoid discomfort or secondary injuries resulting from overload. This is apparently a practical advancement.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 is an assembled perspective view of the present invention.

FIG. 2 is an exploded perspective view of the present invention.

FIG. 3 is a partially enlarged perspective view of the gear assembly of the present invention.

FIG. 4 is an operating state view of the present invention.

FIG. 5 is an unused state view of the driving member and weight member of the present invention.

FIG. 6 is an operating schematic view of the working state of the present invention.

FIG. 7 is an operating schematic view of the driving member and weight member of the present invention.

FIG. 8 is a partially enlarged sectional view of the driving member and weight member shown in FIG. 7.

FIG. 9 is an embodiment view showing a further pivot shaft provided between the front backing portion and rear assembling portion of the present invention.

### DETAILED DESCRIPTION OF THE INVENTION

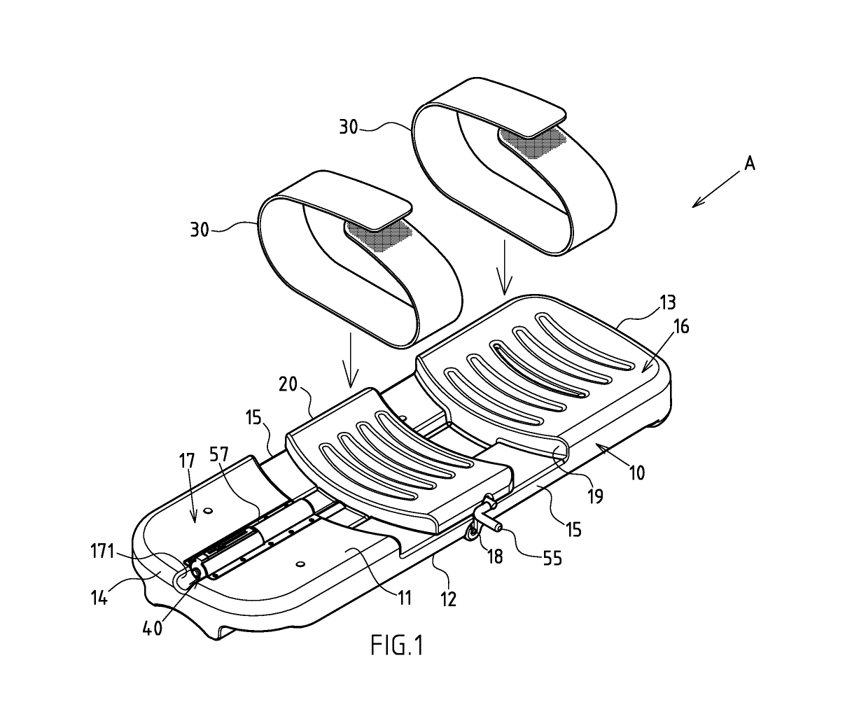

FIGS. 1, 2, 3, and 4 depict a preferred embodiment of the lumbar traction device of the present invention. However, such an embodiment is illustrative only and is not intending to limit the scope of patent claims.

Said lumbar traction device A comprises a tabular base 10, being an integral or assembled plate, including a top surface 11, a bottom surface 12, a front end 13, a rear end 14 and two side parts 15.

An indentation 19 is provided on the top surface 11 of the tabular base 10 and recessed downward and inward, located between the front end 13 and rear end 14 of the tabular base 10, so that the tabular base 10 is formed with a front backing portion 16 and a rear assembling portion 17.

A lumbar tracking base 20 is provided inside the indentation 19 and able to slide forward and backward along the indentation 19.

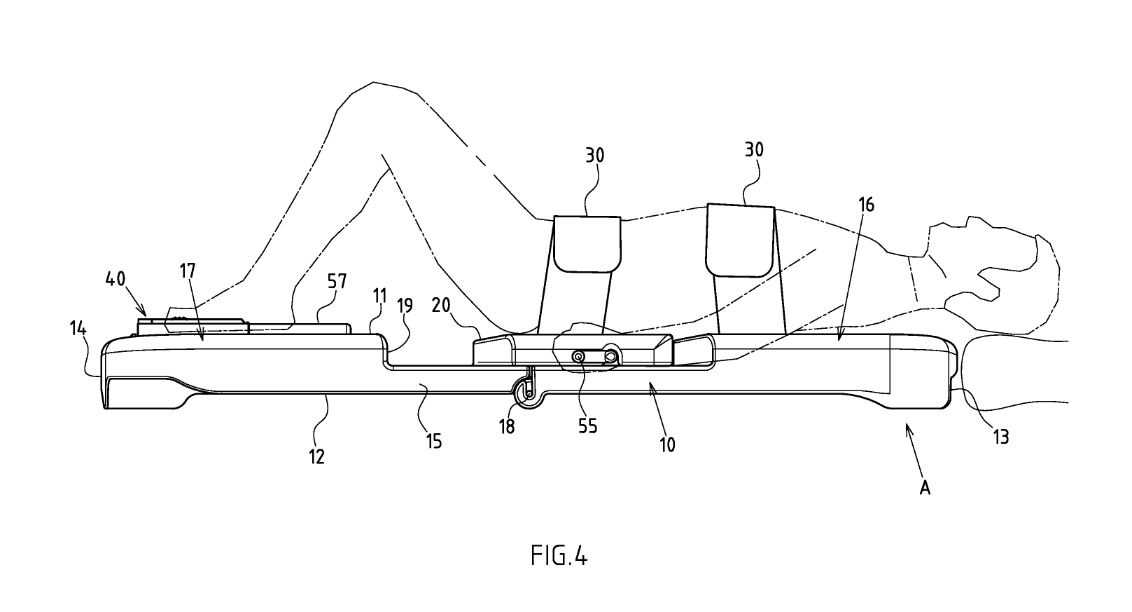

At least two tightening pieces 30 are respectively attached to the lumbar tracking base 20 and front backing portion 16, so as to fix the chest-back part, and lumbar part of the user respectively on the front backing portion 16 and lumbar tracking base 20. Referring to FIG. 1, a preferred embodiment of said tightening pieces 30 can be, but not limited to, a tightening belt. Referring to FIG. 4, said tightening pieces 30 tightens and fixes the user's back part on the front backing portion 16, and the lumbar part on the lumbar tracking base 20, so that when the lumbar tracking base 20 slides to have stretching or releasing movements, only the lumbar part is stretched and pulled, while the user's chest-back part will not move along. This helps to achieve the rehabilitation and curing functions and purposes.

A weight member 40 is assembled on the rear assembling portion 17 of the tabular base 10, the weight member 40 comprises a fixed outer base 41, a sliding inner base 42 pivoted inside the fixed outer base 41, able to slide and protrude front the front end of the fixed outer base 41, wherein a flexible weight piece 43 is configured between the fixed outer base 41 and sliding inner base 42, to provide the weight force required to stretch the lumbar.

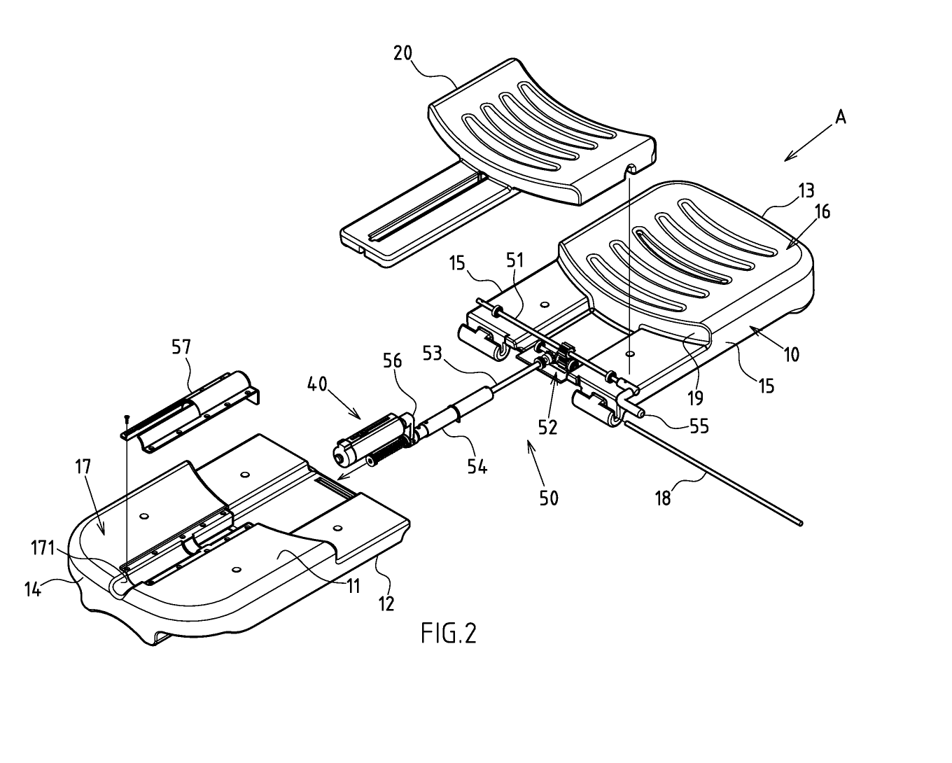

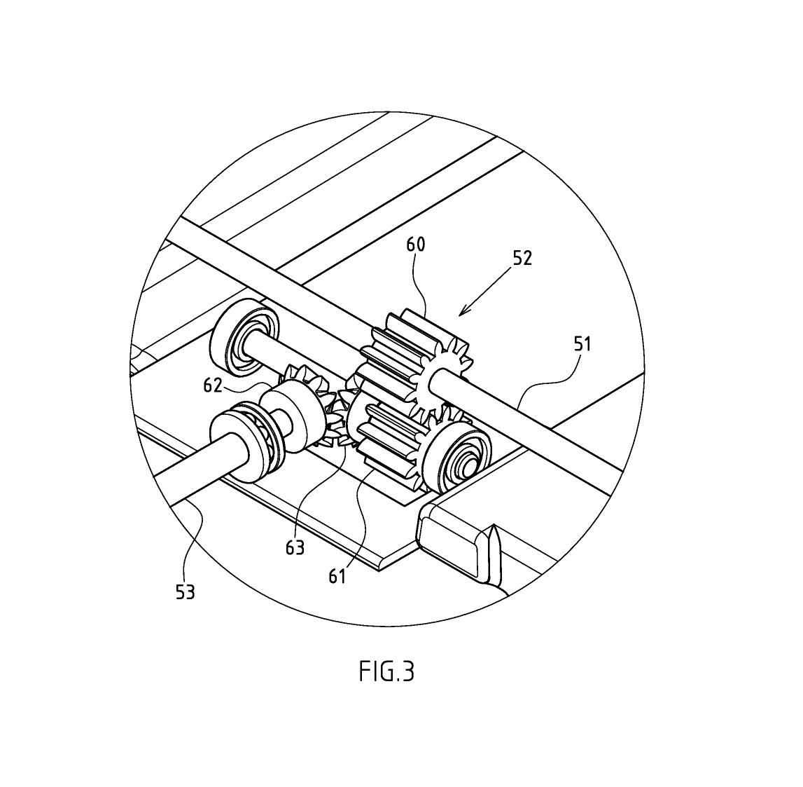

A driving member 50 is provided including a rotation axis 51 extending to the two sides of the tabular base 10, a gear assembly 52, a threaded rod 53 extending to the front and rear ends, and a guide screw tube 54, wherein the guide screw tube 54 of the driving member 50 is connected to the front end of the sliding inner base 42 of the weight member 40, the rotation axis 51 and threaded rod 53 are pivoted on the bottom of the lumbar tracking base 20, the rotation axis 51 and threaded rod 53 are linked through a gear assembly 52 so they are engaged and driven by each other, and the rear end of the threaded rod 53 is screwed into the guide screw tube 54. Referring to FIG. 2, as an implementation method, the rotation axis 51 can be pivoted on the axis hole provided on the bottom of the lumbar tracking base 20 through the bearing attached to the rotation axis 51, that the rotation axis 51 can rotate, while the rear end of the threaded rod 53 is screwed into the guide screw tube 54, and its front end extends to and is pivoted on the bottom of the lumbar tracking base 20 and through the gear assembly 52, it is engaged with and driven by the rotation axis 51. Referring to FIGS. 2 and 3, the gear assembly 52 of the driving member 50 can include a first gear 60 provided on the middle section of the rotation axis 51, a second gear 61 which meshes with the first gear 60, a first bevel gear 62 provided on the front end of the threaded rod 53 and a second bevel gear 63 provided on one end of the second gear 61, and the first bevel gear 62 meshes with the second bevel gear 63, therefore, when the rotation axis 51 is driven to rotate, the first gear 60 can rotate synchronously, and drive the second gear 61 and second bevel gear 63 to drive the first bevel gear 62 and threaded rod 53 to rotate, and the threaded rod 53 can move spirally forward and backward relative to the guide screw tube 54, and consequently, the lumbar tracking base 20 can synchronously move to cause the stretching or releasing functions.

A controller 55 is provided on at least one side of the rotation axis 51, to control the rotation of the rotation axis 51. Referring to FIGS. 1 and 2, the controller 55 can be made of an operating crank provided on at least one side of the rotation axis 51, and through this, the user can rotate the operating crank to drive the rotation axis 51 to rotate.

Referring to FIGS. 1 and 2, the rear assembling portion 17 of the tabular base 10 is further provided with an elongated recession 171, and the guide screw tube 54 of the driving member 50 can be connected to the front end of the sliding inner base 42 of the weight member 40 through a vertical connecting seat 56, so that the weight member 40 and guide screw tube 54 are distributed up and down with some space, further a positioning frame seat 57 is used to lock the weight member 40 and position it inside the elongated recession 171 of the rear assembling portion 17.

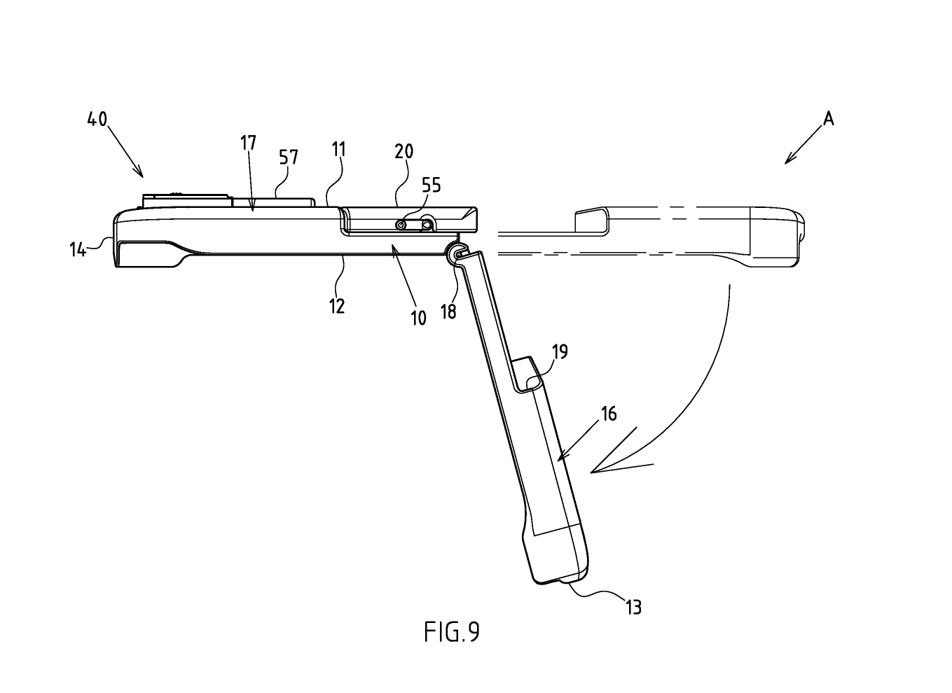

Referring to FIG. 9, between the front backing portion 16 and rear assembling portion 17 of the tabular base 10, a pivot shaft 18 can be provided to form a pivoting state, so that the front backing portion 16 and rear assembling portion 17 can be folded onto each other to reduce space.

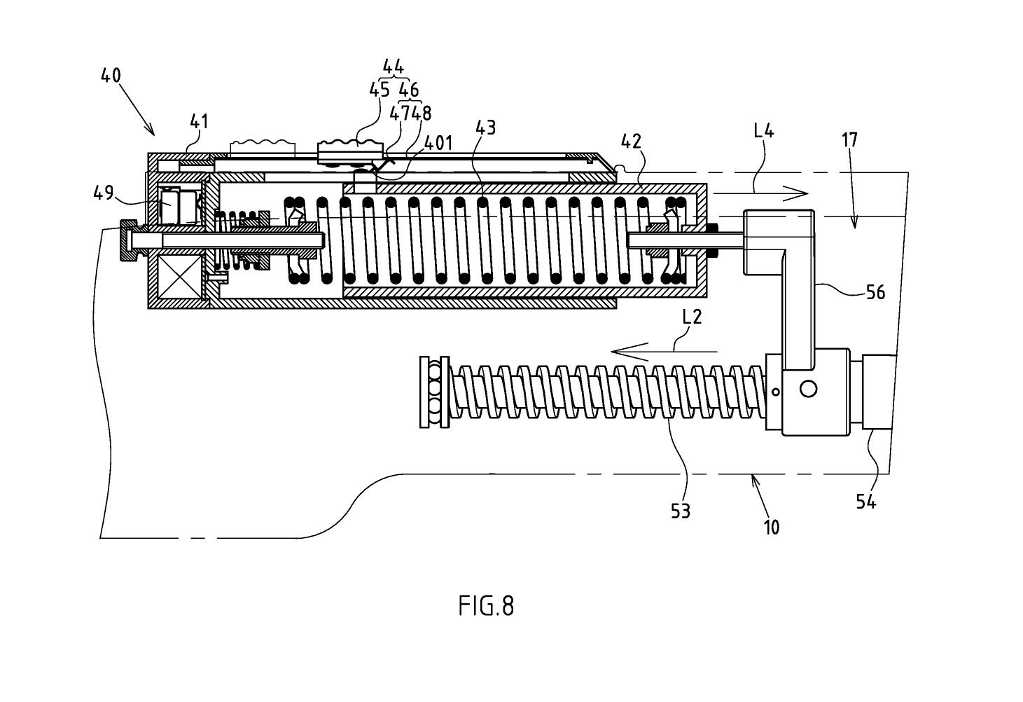

Referring to FIG. 8, one side of the weight member 40 can be further provided with a weight adjustment component 44. The weight adjustment component 44 comprises an adjusting switch 45 and a driving member 46. Said weight adjustment component 44 is preset with the weight value required to stretch the lumbar, and one side of the weight member 40 is formed with a trigger 401. The driving member 46 of the weight adjustment component 44 includes a circuit connecting-disconnecting spring 47 and an adjusting circuit board 48, so that when the trigger 401 provided on the weight member 40 presses on the circuit connecting-disconnecting spring 47, the circuit connecting-disconnecting spring 47 will instantly contact the adjusting circuit board 48 to achieve the preset weight force. Further, on a preset position of the weight member 40, a warning part 49 is provided. When the flexible weight piece 43 of the weight member 40 reaches the weight value preset by the user, the warning part 49 will emit a warning signal. The warning part 49 can be a buzzer, a luminous device or any other device with a warning function, to emit light or sound to remind the user, so as to avoid overload which may cause discomfort or secondary injury.

Based on the structural design described above, the present invention operates as follow:

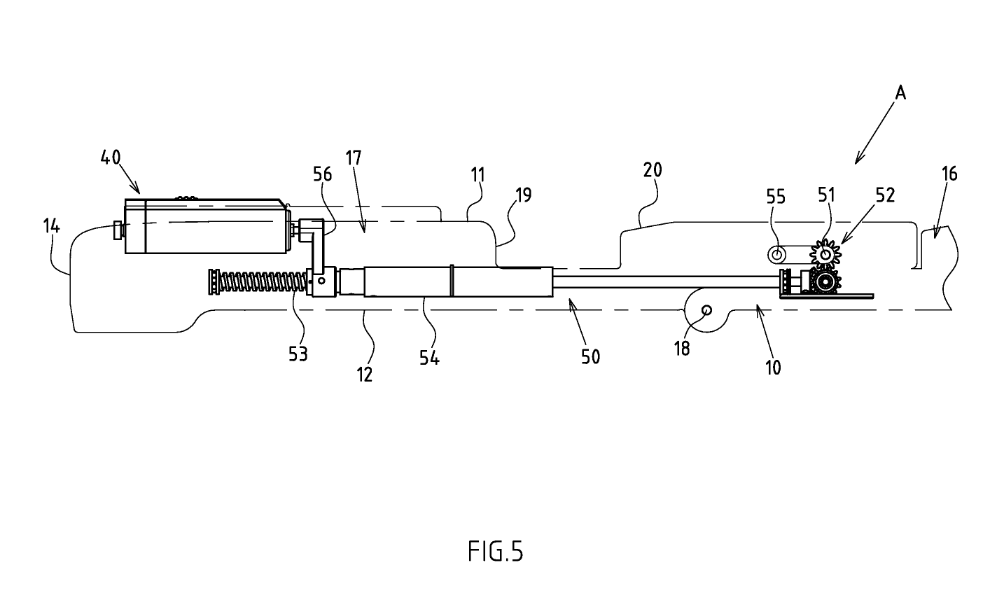

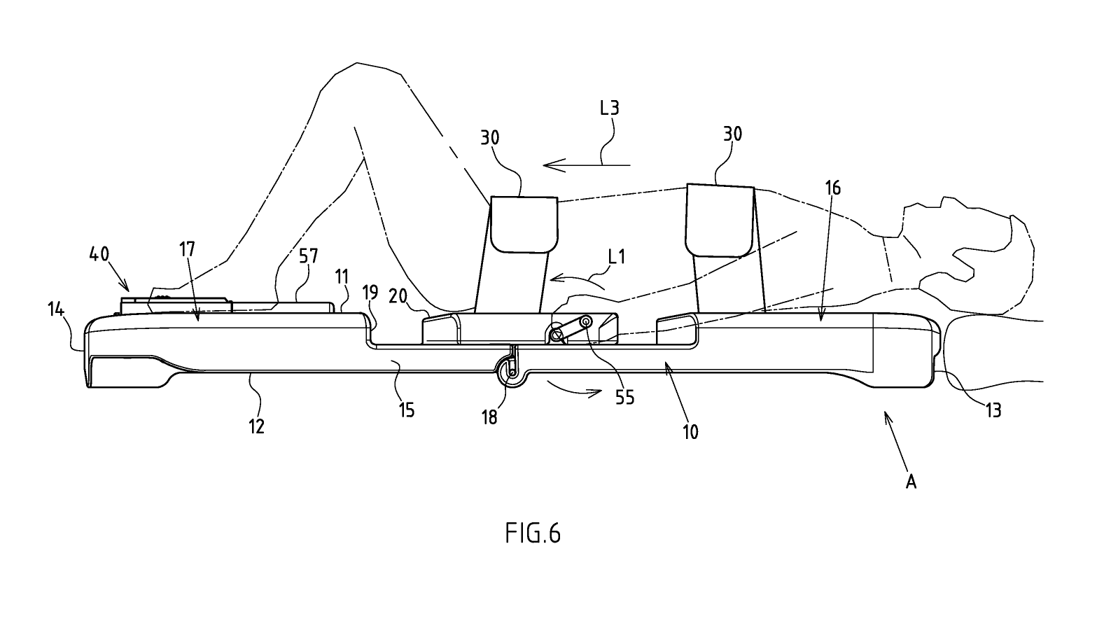

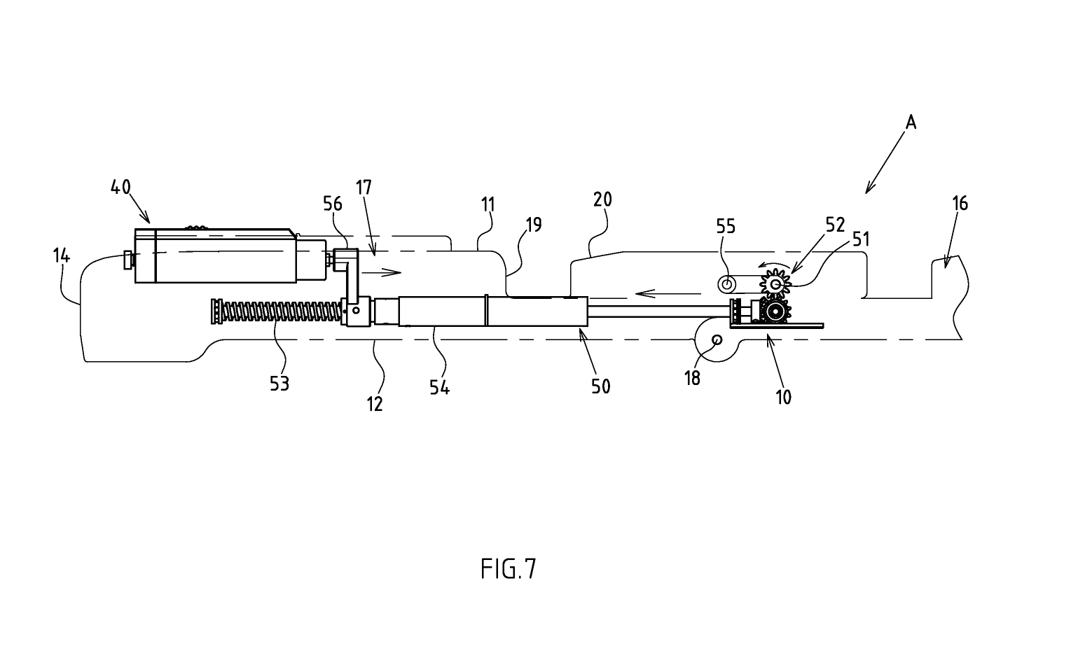

Referring to FIG. 4, the user can lie on the tabular base 10, with the back part leaning on the front backing portion 16, and the lumbar part contacting the lumbar tracking base 20, and use the tightening pieces 30 to tighten the chest-back part and lumbar part respectively onto the front backing portion 16 and lumbar tracking base 20. Now, the driving member 50 and the weight member 40 provided on the tabular base 10 are in an unused state, as shown in FIG. 5. Then, referring to FIG. 6, the user can grip the controller 55 to control the rotation axis 51 to rotate (as indicated by Arrow L1), to drive the threaded rod 53 to rotate spirally backward and synchronously drive the lumbar tracking base 20 to slide backward (refer to FIG. 7), to pull the user's lumbar part backward (as indicated by Arrow L3), so as to achieve the rehabilitation and curing function and purpose. Similarly, if the user grips the controller 55 and rotate forward, the threaded rod 53 will rotate spirally forward to achieve the releasing function. In this way, the user can easily control the lumbar traction device for optimal effect and safety.

Referring to FIG. 8, another objective and innovative technical characteristic of the present invention is that the lumbar traction device A is further provided with a weight member 40, and the weight member 40 comprises a weight adjustment component 44 and a warning part 49, so that the user can preset a required weight value through the adjusting switch 45 provided on the weight adjustment component 44 (for example, a weight value recommended by the doctor, or an adjusted weight value based on individual body weight or enduring ability). In this way, when the user drives the threaded rod 53 to rotate spirally backward through the controller 55 (as indicated by Arrow L2), counter force will be generated to pull the flexible weight piece 43 and move the trigger 401 and sliding inner base 42 forward (as indicated by Arrow L4) to resist against the weight force, when the trigger 401 presses on the circuit connecting-disconnecting spring 47, the circuit connecting-disconnecting spring 47 will contact the adjusting circuit board 48 to achieve the preset weight force. By this time, the warning part 49 will emit a warning signal to inform the user, so as to effectively avoid overload to cause discomfort or secondary injury.

1. A lumbar traction device, comprising:
a tabular base, being an integral or assembled plate, including a top surface, a bottom surface, a front end, a rear end and two side parts;
a indentation, provided on the top surface of the tabular base and recessed downward and inward, located between the front end and rear end of the tabular base, so that the tabular base is formed with a front backing portion and a rear assembling portion;
a lumbar tracking base, provided inside the indentation and able to slide forward and backward along the indentation;
at least two tightening pieces, respectively attached to the lumbar tracking base and front backing portion, so as to fix the chest-back part and lumbar part of the user respectively on the front backing portion and lumbar tracking base;
a weight member, assembled on the rear assembling portion of the tabular base, the weight member comprises a fixed outer base, a sliding inner base pivoted inside the fixed outer base, able to slide and protrude from the front end of the fixed outer base, wherein a flexible weight piece is configured between the fixed outer base and sliding inner base, to provide the weight force required to stretch the lumbar;
a driving member, including a rotation axis extending to the two sides of the tabular base, a gear assembly, a threaded rod extending to the front and rear ends, and a guide screw tube, wherein the guide screw tube of the driving member is connected to the front end of the sliding inner base of the weight member, the rotation axis and threaded rod are pivoted on the bottom of the lumbar tracking base, the rotation axis and threaded rod are linked through a gear assembly so they are engaged and driven by each other, and the rear end of the threaded and is screwed into the guide screw tube;
a controller, provided on at least one side of the rotation axis, to control the rotation of the rotation axis;
in this way, the user can grip the controller to control the rotation axis to rotate, and drive the threaded rod to rotate spirally forward and backward relative to the guide screw tube, and synchronously drive the lumbar tracking base to move forward and backward for the stretching or releasing functions.

2. The device defined in claim 1, wherein the rear assembling portion of the tabular base is further provided with an elongated recession extending to the front and rear ends, and the guide screw tube of the driving member is connected to the front end of the sliding inner base of the weight member through a vertical connecting seat, so that the weight member and guide screw tube are distributed up and down with some space, further a positioning frame seat is used to lock the weight member and position it inside the elongated recession of the rear assembling portion.

3. The device defined in claim 2, wherein the gear assembly of the driving member includes a first gear provided on the middle section of the rotation axis, a second gear which meshes with the first gear, a first bevel gear provided on the front end of the threaded rod and a second bevel gear provided on one end of the second gear, and the first bevel gear meshes with the second bevel gear, therefore, when the rotation axis is driven to rotate, the first gear can rotate synchronously, and drive the second gear and second bevel gear to drive the first bevel gear and threaded rod to rotate, and the threaded rod attached to the weight member can move forward and backward relative to the guide screw tube.

4. The device defined in claim 1, wherein, between the front backing portion and rear assembling portion of the tabular base, a pivot shaft is provided to form a pivoting state, so that the front backing portion and rear assembling portion can be folded onto each other.

5. The device defined in claim 1, wherein, one side of the weight member is further provided with a weight adjustment component; the weight adjustment component comprises an adjusting switch and a driving member; said weight adjustment component is preset with the weight value required to stretch the lumbar, and one side of the weight member is formed with a trigger; and the driving member of the weight adjustment component includes a circuit connecting-disconnecting spring and an adjusting circuit board, so that when the trigger provided on the weight member presses on the circuit connecting-disconnecting spring, the circuit connecting-disconnecting spring will instantly contact the adjusting circuit board to achieve the preset weight force; further, on a preset position of the weight member, a warning part is provided; when the flexible weight piece of the weight member reaches the weight value preset by the user, the warning part will emit a warning signal; and the warning part is either a buzzer or a luminous device.
